(19) United States
(12) Patent Application Publication
(10) Pub. No.: US 2023/0069954 A1
(43) Pub. Date: Mar. 9, 2023

(54) MEDICAL COMPOSITION OR DEVICE COMPRISING OLIGO(ETHYLENE GLYCOL) POLYMERS

(71) Applicants: LVMH RECHERCHE, SAINT JEAN DE BRAYE (FR); URGO RECHERCHE INNOVATION ET DEVELOPMENT, Chenove (FR); UNIVERSITE DE PAU ET DU PAYS DE L'ADOUR, PAU CEDEX (FR); CENTRE NATIONAL DE LA RECHERCHE SCIENTIFIQUE, PARIS (FR)

(72) Inventors: Eva DIEUZY, Grenoble (FR); Laurent BILLON, St Faust (FR); Christophe DERAIL, CESCAU (FR); Valérie ALARD, SAINT JEAN DE BRAYE (FR); Kamel CHOUGRANI, SAINT JEAN DE BRAYE (FR)

(21) Appl. No.: 17/784,507

(22) PCT Filed: Nov. 27, 2020
(86) PCT No.: PCT/FR2020/052213

§ 371 Date: Nov. 21, 2022

(30) Foreign Application Priority Data
Dec. 16, 2019 (FR) 1914551

(51) Int. Cl. A61K 8/81; A61K 8/04; A61Q 19/00; A61K 47/32; A61K 9/06

(52) U.S. Cl. CPC A61K 8/8152; A61K 8/042; A61K 9/06; A61K 47/32; A61Q 19/00; A61K 2800/10; A61K 2800/412

(57) ABSTRACT

The present invention relates to a cosmetic or pharmaceutical composition for topical application, said composition comprising the combination of a crosslinked oligo(ethylene glycol) based polymer in the form of an aqueous dispersion of microgel particles and a non-crosslinked water-soluble oligo(ethylene glycol) polymer. The invention also relates to a process for preparing this composition.

### TECHNICAL FIELD

The present invention relates to a cosmetic, a pharmaceutical composition or a medical device for topical application or application on the mucosa, said composition comprising the combination of a polymer based on crosslinked oligo(ethylene glycol) in the form of an aqueous dispersion of colloidal microgel particles and a non-crosslinked water-soluble oligo(ethylene glycol) polymer. The invention also relates to a process for preparing this composition.

### PRIOR ART

In the field of adhesive polymers, the modification of the viscoelastic modulus is mainly achieved by the addition of tacky resins or oils, which require complete miscibility with the polymer (Tse MF, Jacob L. Pressure Sensitive Adhesives Based on VectorR SIS Polymers I. Rheological Model and Adhesive Design Pathways. The Journal of Adhesion. 1996 Apr 1; 56(1-4):79-95, and C. Derail, A. Allal, G. Marin, Ph. Tordjeman, Relationship between viscoelastic and peeling properties of model adhesives. Part 1: Cohesive fracture, J. of Adhesion, 61, 123-157, 1997).

There is a need to propose a novel route for modulating the mechanical properties of cosmetic and pharmaceutical films which are intended to be applied to the mucosa or skin.

In the context of the present invention, we found that compositions based on poly(oligo-(ethylene glycol) methacrylate) microgels achieve this purpose.

The synthesis of aqueous dispersions of such microgels has been described in the literature, in particular in patent applications WO 2016/110615 and WO 2019/077404, and the publications of Boularas et al., Polymer Chem., 2016, 7, 350-363; and Aguirre et al., Polymer Chem., 9, 1155-1159. During the synthesis of these microgels, a by-product is formed, which consists of a water-soluble polymer (WSP), referred to as a free polymer. By evaporation of the aqueous solvent, the self-assembly of the purified microgels forms cohesive and elastic films.

There is a need to propose cosmetic products, pharmaceutical products and medical devices that are intended to be applied on the skin or the mucosa, said films being sufficiently flexible, cohesive and adhesive, in order to stay in place once applied without breaking, crumbling or tearing. It is also desirable that these products be removable by hand without tearing and without leaving residue on the skin.

However, the inventors discovered that the mixture of purified microgels with the free polymer makes it possible to direct and control the viscoelastic modulus value of the films without reducing the elongation at break. In particular, the free polymer makes it possible to increase the adhesion of a microgel film without changing its mechanical properties.

The addition of a free polymer at different concentrations makes it possible to direct the rheological and mechanical properties of the films and, consequently, the tack, in other words, stickiness.

In the present invention, the films obtained by drying the association of microgels and free polymer have particular mechanical properties of elasticity and stickiness. The inventors surprisingly found that these properties could be modulated depending on the microgel-polymer mass ratio. The inventors discovered that the chemical composition of the free polymer is very comparable to that of the microgels and permits good compatibility between the two components. Moreover, the branched and crosslinked structure allows conserving the resistance to stretching of the films obtained from their mixtures.

By evaporating the aqueous solvent, self-assembly of the microgel/polymer mixture forms a film with promising mechanical properties for cutaneous applications: the elastic modulus is low and the deformability and elongation at break are high. The reformulation with the free polymer potentially makes it possible to direct and control the viscoelastic modulus value of the films without reducing the elongation at break.

The formation of microgel films with adjustable mechanical properties has never been related in the literature. In this invention, the variation of the rheological properties of the films is directly obtained thanks to a by-product of synthesis, the free polymer. This has the same chemical composition as the microgels and therefore has an excellent compatibility with these microgels. This has the advantage of not weakening the self-assembled microgel network, which is reflected by the preservation of the resistance to stretching.

The addition of the free polymer can induce a variation of the real part of the complex shear modulus (G′) between 1x10³ and 1x10⁵ Pa. This makes it possible to obtain films with a very pronounced tackiness up to films with no stickiness at all. However, the elongation at break, which reflects the cohesion of the microgel network at very high deformations, does not decrease with the addition of the free polymer. The structural characterization of the free polymer by aqueous chromatography reveals a highly branched polymer resembling nanogels, whose structure explains the conservation of the resistance of the network which can thus follow the deformations of the skin or the mucosa without detaching or breaking.

### GENERAL DESCRIPTION OF THE INVENTION

A first subject-matter of the invention is a composition, especially a cosmetic composition, a pharmaceutical composition or a medical device, comprising a water-soluble polymer (WSP) and microgel particles, in which the microgel particles and the water-soluble polymer can be independently obtained, or are independently obtained, by aqueous phase precipitation polymerization of di(ethylene glycol) methyl ether methacrylate, an oligo(ethylene glycol) methyl ether methacrylate, and a vinyl monomer bearing a carboxyl group in the presence of N,N′-methylenebisacrylamide as crosslinking agent, in which the ratio between the mass of the water-soluble polymer and the mass of the microgel particles in the dry state is between 0% and 100%.

According to one embodiment, the ratio between the mass of the water-soluble polymer and the mass of the microgel particles is between 0% and 35% or between 35% and 100%.

A second subject-matter of the invention is a process for cosmetic treatment or pharmaceutical treatment by topical route of the skin, nails, lips, mucosa or hair of a person, said process comprising a first step of applying the cosmetic composition, the pharmaceutical composition or the medical device as defined above.

A third subject-matter of the invention is a process for preparing the cosmetic composition, the pharmaceutical composition or the medical device, said process comprising a step of preparing a mixture of microgel particles and a water-soluble polymer, said step comprising:
a first step of preparing microgel particles,
a second step of preparing the water-soluble polymer, and
a third step of mixing microgel particles and water-soluble polymer, the ratio between the mass of the water-soluble polymer and the dry mass of the microgel particles being between 0% and 100%.

### DESCRIPTION OF THE FIGURES

FIG. 1 is a conformation graph of the radius of gyration as a function of molar mass for MBA-WSP (red), OEGDA-WSP (blue) and PEG35K (black). WSP is synthesized with 2 mol% of crosslinker. The dotted line is representative of a power law with an exponent of 0.6. The solid black line fits the MBA-WSP curve according to a power law of exponent 0.27.

In FIG. 2, the number and weight average molecular weights, and the polydispersity index for different WSPs are indicated.

FIG. 3 represents the curve of the conservation modulus G' and of the loss modulus G" as a function of the frequency of an unpurified MBA film and a unpurified OEGDA film with different amounts of crosslinking agent: 2 mol% and 8 mol%.

FIG. 4 is a table providing the mass composition of unpurified films, after synthesis.

FIG. 5A represents the extensional viscosity as a function of time of unpurified MBA films and OEGDA films with different amounts of crosslinker: 2 mol% and 8 mol%.

FIG. 5B is the elongation at break for an MBA film and an OEGDA film for different crosslinking densities at 2 mol% and 8 mol%.

FIG. 6A represents the conversion modulus G' and the loss modulus G" as a function of the frequency of a 2.0 mol% OEGDA-MG film with different amounts of MG.

FIG. 6B is the conversion modulus G' and the loss modulus G” as a function of the frequency of a 2.0 mol% MBA-MG film with different amounts of MG.

FIG. 7 presents G′ (square), G″ (triangle) and tan δ (circle) as a function of the microgel content at ω = 0.01 rad.s⁻¹. The solid symbols correspond to OEGDA and the hollow symbols correspond to MBA.

FIG. 8A is the extensional viscosity curve as a function of time for OEGDA-MG films at different MG contents.

FIG. 8B is the extensional viscosity curve as a function of time of OEGDA-MG films for different MG contents.

FIG. 9 is a bar chart representing the elongation at break for an MBA film and an OEGDA film for different MG contents.

FIG. 10A represents the extensional viscosity curve as a function of time for pure OEGDA-MG films at different film formation temperatures.

FIG. 10B is a histogram showing the elongation at break values for pure OEGDA-MG films at different film formation temperatures.

FIG. 11 shows two slides obtained by atomic force microscopy (AFM) of the upper surface of OEGDA-MG films (left) and MBA-MG films (right).

FIG. 12 shows two AFM slides of a cross section of OEGDA-MG films (left) and MBA-MG films (right).

FIG. 13 shows two slides obtained by atomic force microscopy (AFM) of the upper surface of OEGDA-MG films (left) and MBA-MG films (right) subjected to an elongation of 30%.

FIG. 14 presents AFM in topographic contrast and images of the LogDMT modulus: (a) AFM by topographic contrast and (b) LogDMT modulus for 2 mol% MBA-MG films containing 25% MG, (c) AFM by topographic contrast and (d) LogDMT modulus for 2 mol% MBA-MG films containing 50% MG; (e) AFM by topographic contrast and (f) LogDMT modulus for 2 mol% MBA-MG films containing 75% MG.

### DEFINITIONS

It is understood within the meaning of the invention that “microgel particles” are a cross-linked polymer in the form of spherical particles having an average size which can vary from 100 nm to 1000 nm in the dry state (i.e., containing less than 2% by mass of water), preferably between 100 nm and 500 nm, from 350 to 450 nm and even better 400 nm. The hydrodynamic radial distribution function of the microgels measured at an angle of 60° and at a temperature of 20° C. can be less than 1.1.

The microgel of the invention can be obtained by copolymerization in aqueous phase of several monomers. The mean size of the microgel particles can vary depending on whether they contain water.

“Microgels”, within the meaning of the present invention, can be in the form of an aqueous dispersion of “microgel particles” or in the form of a film comprising microgel particles as defined above. Microgels can trap cosmetically or pharmaceutically active organic molecules. A film comprising microgel particles can have a thickness of 1 micron to 10 millimeters, for example from 10 microns to 500 microns, from 100 microns to 400 microns or from 500 microns to 1000 microns. In a particular embodiment, the microgel particles preferably do not comprise inorganic material.

It is preferred that the microgel particles be made up of organic compounds. The microgel particles do not contain silica, for example, in particular silica as a cross-linked polymer carrier.

A “crosslink” is a group (part of a molecule) which binds copolymer chains together. This crosslink originates from a “crosslinker” molecule which is mixed with the monomers during the process of polymerizing the crosslinked polymer.

Within the meaning of the invention, it is understood that a “water-soluble polymer” is a polymer having a radius of gyration at 20° C. which is from 5 nm to 80 nm, for example, from 10 nm to 30 nm. The water-soluble polymer may have an average molecular weight of 1 x 10⁵ g.mol⁻¹ to 1 x 10⁶ g.mol⁻¹. The radius of gyration and the molecular weight can be measured by any process known to the skilled person, for example by size exclusion chromatography. A water-soluble polymer is distinguished from microgel particles: for example microgel particles can be identified by atomic force microscopy (AFM) observation of a film produced by drying an aqueous dispersion of microgel particles. In contrast, no particles can be detected by AFM observation of films that are produced by drying a solution of water-soluble polymer.

Within the meaning of the invention, the expression “between” excludes the numerical limits that follow it. On the other hand, the expression “from... to” includes the limits mentioned.

### DETAILED DESCRIPTION OF THE INVENTION

The first subject-matter of the invention concerns a composition, especially a cosmetic composition, a pharmaceutical composition, or a medical device, comprising a water-soluble polymer and microgel particles, said microgel particles having an average diameter of 100 nm to 1000 nm in the dry state, in which the microgel particles and water-soluble polymer may be independently obtained, or are independently obtained, by aqueous phase precipitation polymerization of at least the following three monomers, in the presence of a crosslinking agent:
di(ethylene glycol) methyl ether methacrylate,
an oligo(ethylene glycol) methyl ether methacrylate,
a vinyl monomer comprising a carboxyl group,
where at least one of the two crosslinking agents (i.e., at least the crosslinking agent used to prepare the microgel particles or at least the crosslinking agent used to prepare the water-soluble polymer, is N,N′-methylenebisacrylamide and where the ratio between the mass of the water-soluble polymer and the mass of the microgel particles in the dry state is between 0% and 100% by mass.

In the case where the composition constitutes or forms part of a medical device, this device includes a mixture consisting of water-soluble polymer, microgels and, optionally, water.

The solid content of the cosmetic composition, the solid content of the pharmaceutical composition and the solid content of the mixture included in the medical device is preferably 1.5% to 100% by mass.

The solid content can be greater than a percentage chosen in the group consisting of 2%; 3%; 4%; 5%; 10%; 15%; 20%; 25%; 30%; 35%; 40%; 45% and 50% by mass. The solid composition of the invention can be defined as the mass percentage of the solid elements that it contains, in particular as equal to the sum of the mass percentage of the microgels in the dry state, the mass percentage of the water-soluble polymer, and, possibly, the percentage of any other solid compound which may be present in the composition. In the case of a medical device, the solid content can be the sum of the percentage of microgels in the dry state and the percentage of the polymer which are contained in the mixture.

According to one embodiment, the microgel particles and water-soluble polymer comprise chains having monomer units of diethylene glycol methacrylate, monomer units of oligoethylene glycol methacrylate comprising 6 to 10 ethylene glycol motifs, methacrylic acid monomer units, and crosslinks. The oligo(ethylene glycol) methyl ether methacrylate preferably comprises 7 to 8 ethylene glycol motifs.

The oligo(ethylene glycol) methyl ether methacrylate can have a number average molar mass (Mn) from 400 g/mole to 600 g/mole, preferably from 450 to 500 g/mole.

The vinyl monomer can be a monomer of formula CR1R2═CR3R4 wherein R1, R2, R3 and R4 represent a hydrogen, a halogen or a hydrocarbon group, on the condition that at least one of the four groups comprises a -COOH or —COO—M⁺, group, M⁺ representing a cation. The vinyl monomer is preferably a (meth)acrylic acid monomer.

The microgel particles and water-soluble polymer may be independently obtained, or are independently obtained, by aqueous phase precipitation polymerization of three monomers in the presence of a crosslinking agent. The step of precipitation polymerization comprises contacting, in an aqueous phase, the three monomers described above and the crosslinking agent, at a temperature between 40° C. and 90° C., preferably of the order of 70° C. The process does not require the presence of a surfactant such as sodium dodecyl sulfate (SDS), and the polymerization can be initiated by the addition of a water-soluble radical initiator, for example potassium persulfate (KPS).

According to one embodiment, the mole fraction of di(ethylene glycol) methyl ether methacrylate is from 80 mol% to 90 mol%, the mole fraction of oligo(ethylene glycol) methyl ether methacrylate is from 5 mol% to 15 mol%, the mole fraction of the carboxyl-bearing vinyl monomer is from 2 mol% to 8 mol%, and the mole fraction of the crosslinking agent is from 0.5 to 10 mol%, the sum of the four mole fractions being equal to 100% by mole. In the present description, the mole fractions can be defined as the mole fractions of the monomers that are used to prepare the microgel or the water-soluble polymers. The mole fractions can be otherwise defined as the mole fractions of monomer units in the microgel or in the water-soluble polymer which have been obtained from the reaction between monomers.

The mole fraction of crosslinking agent can be from 0.5 mol% to 10 mol%, from 0.5 mol% to 8 mol%, from 1 mol% to 7 mol% or from 1.5 mol% to 6 mol%.

The molar ratio (a:b) between the di(ethylene glycol) methyl ether methacrylate (a) and the oligo(ethylene glycol) methyl ether methacrylate (b) is preferably between 1:1 and 20:1, for example between 5:1 and 10:1.

The (meth)acrylic acid monomer can have the formula CR1R2═CR3R4 wherein R1, R2, R3 and R4 represent a hydrogen, a halogen or a hydrocarbon group, at least one of the four groups comprising a —COOH or —COO—M⁺ group, M⁺ representing a cation.

The (meth)acrylic acid monomer can be chosen from the group consisting of methyl acrylic, methyl methacrylic, ethyl acrylic, ethyl methacrylic, n-butyl acrylic, and n-butyl methacrylic, methacrylic, itaconic or acrylic acids. Methacrylic acid is preferred.

The oligo(ethylene glycol) methyl ether methacrylate can have a molecular weight between 200 g/mole and 600 g/mole, or between 300 g/mole and 550 g/mole between 450 g/mole and 500 g/mole.

In a particular embodiment, the mole fraction of di(ethylene glycol) methacrylate monomer units is from 80 mol% to 90 mol%, preferably from 82 mol% to 86 mol%, the mole fraction of oligo(ethylene glycol) methyl ether methacrylate monomer units is from 5 mol% to 15 mol%, preferably from 7 mol% to 11 mol%, the mole fraction of (meth)acrylic acid monomer units is from 2 mol% to 8 mol%, preferably from 3 mol% to 7 mol%, and the mole fraction of the crosslink is from 1 mol% to 6 mol% or from 1 mol% to 3 mol%.

According to another embodiment, the crosslinked polymer comprises polymer chains having diethylene glycol methacrylate monomer units, oligoethylene glycol methacrylate monomer units comprising 4 to 10 ethylene glycol motifs and methacrylic acid monomer units. The monomer units are preferably: oligo(ethylene glycol) methyl ether methacrylate having 7 or 8 ethylene glycol motifs and methacrylic acid. The oligo(ethylene glycol)methyl ether methacrylate monomer units can also have 9 ethylene glycol motifs.

The microgel is obtained by polymerization of at least three monomers in the presence of a crosslinking agent and the water-soluble polymer is obtained from a polymerization of at least three monomers in the presence of a second crosslinking agent. The first crosslinking agent and/or the second crosslinking agent are N,N′-methylenebisacrylamide.

According to one embodiment, one of the two crosslinking agents has terminal di(meth)acrylate groups and a motif chosen in the group consisting of —(CH₂—CH₂—O)ₘ—CH₂—CH₂— where n is from 0 to 6, —NH—CH₂—NH— and mixtures of these. The number m is preferably from 3 to 6.

One of the crosslinking agents is, for example N,N′-methylenebisacrylamide (MBA) while the other is (ethylene glycol) dimethacrylate (EGDMA) or an oligo(ethylene glycol) diacrylate (OEGDA). According to one embodiment, the first crosslinking agent and the second crosslinking agent are both N,N′-methylenebisacrylamide.

According to a preferred embodiment, the cosmetic composition, the pharmaceutical composition and the mixture included in the medical device are in the form of a film comprising microgels which are obtained with 1.8-2.2 mol% of N,N′-methylenebisacrylamide as crosslinking agent. These films are advantageous because they are very flexible.

In a particular embodiment, the cosmetic composition, the pharmaceutical composition or the mixture which is included in the medical device is in the form of a film which has a thickness of 500 microns to 1000 microns.

The ratio between the mass of the water-soluble polymer and the mass of the microgel particles is greater than 0% and less than 100%. The ratio can have a lower value which is chosen in the group consisting of 5%, 15%, 20%, 25%, 30%, 35%, 40%, 45%, 50%, 55%, 60%, 65%, 70%, 75%, 80%, 85%, 90% and 95%. The ratio can have an upper value which is chosen in the group consisting of 5%, 15%, 20%, 25%, 30%, 35%, 40%, 45%, 50%, 55%, 60%, 65%, 70%, 75%, 80%, 85%, 90% and 95%.

In one embodiment, the ratio between the mass of the water-soluble polymer and the mass of the microgel particles is between 0% and 35%. In a second embodiment, the ratio between the mass of the water-soluble polymer and the mass of the microgel particles is between 35% and 100%.

The cosmetic composition and the pharmaceutical composition can be a liquid, a gel or a solid.

The cosmetic composition, pharmaceutical composition or mixture consisting of the water-soluble polymer, the microgels and, optionally, water which is included in the medical device may contain from 50% to 100% by mass of a mixture consisting of microgel particles and water-soluble polymer and from 0% to 50% by mass of water, the percentages being relative to the mass of the composition and the mass of the microgel particles being the mass of the particles in the dry state. The solid content of the cosmetic composition, the solid content of the pharmaceutical composition and the solid content of the mixture consisting of the water-soluble polymer, the microgels and, optionally, water which is included in the medical device can be from 50% to 100% by mass. In the case where the composition of the invention is liquid, it can be spread on the skin or mucosa and can form a transparent film adhering to the skin or the mucosa by simple evaporation of the water. It is known that the skin has a low modulus of elasticity and that it is necessary that the product applied have a modulus comparable to that of the skin so as not to be felt by the user. In this invention, the free polymer is a lever to adjust the modulus according to the targeted application. When a product is formulated, the addition of components, such as gelling or bioactive agents can increase the modulus; it is then possible to add the suitable proportion of free polymer to reduce it to a target value. The films have a high elongation at break regardless of the amount of free polymer, which is vital for application on the face, for example.

The composition can comprise 1% to 100% by mass of microgels. According to one embodiment, the composition comprises from 1% to 50% by mass of microgels and from 0.9% to 100% by mass of water relative to the mass of the composition.

The composition can be in the form of a dispersion of microgels in water. The aqueous dispersion can comprise from 1% to 50% by mass of a mixture consisting of water-soluble polymer gels on the basis of the mass of the composition, and from 50% to 99% by mass of water, on the basis of the mass of the composition.

The composition can be in the form of a film having a thickness of 1 micron to 10 millimeters comprising from 50% to 100% by mass of a mixture consisting of microgels and water-soluble polymer, and from 0% to 50% by mass of water, the percentages being expressed relative to the total mass of the composition.

The films can be produced from an aqueous dispersion comprising the microgel and the water-soluble polymer by evaporation of water. The aqueous dispersion is, for example, spread over a rigid or flexible substrate, where the substrate has a temperature between 20° C. and 60° C., even better a temperature from 30° C. to 40° C., even better still a temperature equal to 35° C.

The films can be formed by a step consisting of placing in a mold a dispersion of microgel particles in water and the water-soluble polymer in water and by a step of drying the dispersion. Drying can be done by placing the mold at a temperature greater than room temperature, for example a temperature of from 30° C. to 60° C.

The cosmetic composition can comprise at least one component chosen in the group consisting of preservatives, fragrances, emollients, surfactants, oils, biologically-active products, pigments and dyes.

According to the second subject-matter of the invention, the process of the invention comprises a first step of preparing the microgels, a second step of preparing the water-soluble polymer and a third step of mixing the two.

The process for preparing a cosmetic composition, a pharmaceutical composition or a medical device such as described previously can comprise a step of preparing a mixture of microgel particles and a water-soluble polymer, said step comprising a first step and a second step which can be successive in any order or simultaneous:
said first step being a step of preparing the microgel particles and comprising a step (i) of aqueous phase precipitation polymerization of at least the following three monomers, in the presence of a crosslinking agent:
di(ethylene glycol) methyl ether methacrylate,
an oligo(ethylene glycol) methyl ether methacrylate,
a vinyl monomer bearing a carboxyl group,
and a purification step (ii) to recover the microgel particles which have been obtained from step (i),
said second step of preparing the water-soluble polymer comprising a step (a) of aqueous phase precipitation polymerization of at least the following three monomers, in the presence of a crosslinking agent:
di(ethylene glycol) methyl ether methacrylate,
an oligo(ethylene glycol) methyl ether methacrylate,
a vinyl monomer bearing a carboxyl group,
and a purification step (b) to recover the water-soluble polymer which has been obtained from step (a),
in which at least the first or the second crosslinking agent is N,N′-methylenebisacrylamide,
   a third step which follows said first step and said second step, said third step being a step of mixing a mass of purified microgel particles which have been obtained from the first step and a mass of water-soluble polymer which is obtained from the second step,
   where the ratio between the mass of the water-soluble polymer and the dry mass of the microgel particles is between 0% and 100%.

Step (ii) can comprise at least one centrifugation/redispersion cycle to separate the precipitate and the supernatant material, and a step of recovering the microgel particles in the precipitate.

Likewise, step (b) can comprise at least one centrifugation/redispersion cycle to separate the precipitate and the supernatant material, and a step of recovering the water-soluble polymer in the supernatant material.

According to one embodiment, the first step and the second step are a single step, such that microgel particles and a water-soluble polymer are produced at the same time from the same monomers, in which the single step comprises at least one centrifugation/redispersion cycle for separating the precipitate and the supernatant material which have been obtained from the aqueous phase precipitation polymerization of the monomers, said microgel particles being recovered in the precipitate and said water-soluble polymer being recovered in the supernatant material.

In the case where the first step and the second step are a single step - the ratio between the mass of the water-soluble polymer in the supernatant material and the mass of the microgel particles in the precipitate is a first ratio and - in the third step - the ratio between the mass of the water-soluble polymer and the mass of the microgel particles is a second ratio which is between 0% and 100% and which is different from the first ratio.

The first crosslinking agent and the second crosslinking agent are preferably both N,N′-methylenebisacrylamide, although one of them can be different therefrom.

The invention also concerns a process for cosmetic treatment or pharmaceutical treatment by topical route of the skin, nails, lips, mucosa or hair of a person, said process comprising a first step of applying onto the person a composition or medical device as described above. The process can comprise a second step of drying the composition or device which has been applied onto the person to obtain a flexible, cohesive and adhesive film.

### EXAMPLES

Poly(oligo-(ethylene glycol) methacrylate) microgels, water-soluble polymers and films comprising the mixture of these microgels and water-soluble polymers are prepared according to the following protocol. Their rheology was studied (linear and non-linear) and their structure was observed by atomic force microscopy (AFM).

### A) Preparation of Microgels (MG), Water-Soluble Polymers (WSP) and Films Comprising These

### Starting Materials

Di(ethylene glycol) methyl ether methacrylate (MEO2MA, 95%), oligo(ethylene glycol) methyl ether methacrylate (OEGMA, terminated by 8 EG units with Mn=475 g.mol-¹), methacrylic acid (MAA), poly(ethylene glycol) diacrylate (OEGDA, Mn=250 g.mol⁻¹), N,N-methylenebisacrylamide (MBA) and potassium persulfate (KPS) were purchased from Sigma Aldrich and used as received. Purified water was used with a Millipore Milli-Q system.

### Microgel Synthesise, WSP Synthesis and Film Preparation

MEO2MA (92.6 mmol), OEGMA (10.3 mmol) and a crosslinker (OEGDA or MBA) were dissolved in 930 g of water. The crosslinker ratios are set at either 2.0 mol% or 8.0 mol% depending on the total of vinyl molecules, respectively corresponding to 2.12 mmol and 9.42 mmol. The mixture is introduced into the 2-L reactor and stirring is set at 150 rpm. The reactor is purged with nitrogen for 45 min to eliminate oxygen at ambient temperature. MAA (5.41 mmol) is dissolved in 30 g of water and added to the reactor. The mixture is then heated to 70° C. Finally, KPS (0.958 mmol) is dissolved in 40 g of water and inserted into the reactor to start the reaction. The reaction is finally maintained at 70° C. for 6 hours.

A first part of the aqueous suspension is obtained comprising MG and WSP and the films (F6, F6', F7 and F7') are formed.

A second part of the aqueous suspension is separated by 3 centrifugation cycles 20,000 rpm, 20 min) where WSP is maintained in the aqueous supernatant material while MG is found in the precipitate. Films with WSP alone (F5 and F5') films with MG alone (F1 and F1') are formed as comparative films.

Films comprising a mixture of WSP and MG in a predetermined ratio are also prepared (F2, F2', F3, F3', F4 and F4').

### Process for Preparing the Films

Films were formed by direct evaporation of water from microgel solutions in a glass bell furnace heated to 37° C. It was demonstrated that the formation temperature does not influence the film properties. Silicone molds were used as containers for easy removal of the films after drying. For the rheological experiments, the final film thicknesses are between 500 microns and 1000 microns. All the thicknesses below and above these values are nevertheless technically feasible, for example as thin as 500 nm.

### B) Determination of the Molar Mass of the Water-Soluble Polymer (WSP) by Size Exclusion Chromatography

- Method: The steric exclusion chromatography (SEC) apparatus consists of a set of aqueous columns from Shodex and an Agilent 1260 Iso pump from Agilent Technologies. The apparatus is connected with a multi angle light scattering (MALS) detector and a differential refractometer detector (RI). The MALS detector used is a Dawn Heleos detector from WYATT Technology. The RI detector is an Optilab T-rEX from WYATT Technology operating at a laser wavelength of 664 nm. The flow rate is set at 0.5 mL/min during the experiment and the column temperature is set at 30° C. The mobile phase consists of a 0.1 g/mole solution of NaNO₃ (8.2 g/L) and sodium azide NaN₃ (0.1 mol/L) as eluent, stabilized with a pH 8 buffer. The mobile phase is filtered before use at 0.1 µm. The water-soluble polymer solutions are prepared at a concentration of 200 ppm in a pH 8 buffer, of which a volume of 100 µL is injected. The solutions are filtered before use at 250 nm to eliminate any possible impurities and microgels.

To interpret the SEC results, the value of the refractive index increment (dn/dc) is experimentally measured on an Optilab T-rEX refractometer from WYATT Technology with a laser wavelength of 532 nm. Five solutions of WSP are prepared with Milli-Q water at different concentrations (0.97 g.L-¹; 0.75 g.L⁻¹; 0.51 g.L⁻¹ and 0.11 g.L⁻¹).

- Results: Steric exclusion chromatography allows determining the molar mass of WSP from a 2 mol% OEGDA synthesis and a 2 mol% MBA synthesis. This technique has the advantage of measuring both the molecular weight and the radius of gyration. It thus provides more information regarding the WSP structure. OEGDA-WSP and MBA-WSP are compared to a linear PEG with an average molecular weight of 35,000 g.mol⁻¹. The molar mass and radius of gyration are linked by Flory theory.

With an SEC technique, separation is done by size: larger objects will pass through the column set sooner (=in a smaller elution volume) than smaller objects.

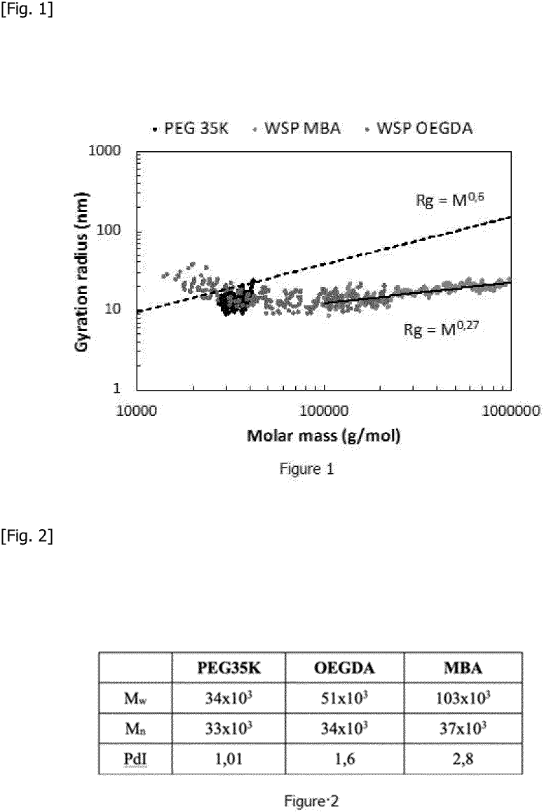

FIG. 1 shows the conformation graph, i.e. the radius of gyration, as a function of molecular weight for OEGDA-WSP, MBA-WSP and PEG 35K. FIG. 2 summarizes the molecular weights and polydispersity index of OEGDA-WSP, MBA-WSP and PEG 35K. PEG 35K has an average Mw at 34,000 g.mol⁻¹ which validates the reliability of the method used. Since the polymer has a very narrow mass distribution, it is not possible to observe the variation of the radius of gyration as a function of the molecular weight. OEGDA-WSP and MBA-WSP have in common a similar population of molecular weights from 1 x10⁴ to 1 x 10⁵ g.mol⁻¹. These polymer chains have a radius of gyration at around 15-20 nm on average, which shows that they have a very dense structure. MBA-WSP has a significant and substantial proportion of much higher molecular weights of from 1 x10⁵ to 1 x10⁶ g.mol⁻¹, which does not appear in the OEGDA chromatogram. These weights reach radii of gyration of up to 30 nm. The detection limit of the equipment corresponds to Rg equal to around 10 nm and explains the dispersed value for the lowest radii. As stated, the linear PEG 35K (black dots) has a weight distribution so narrow that it is not possible to adjust the radius of gyration by a power law. The black line drawn for Rg = 0.6 Mw was thus added to represent a theoretical linear polymer in a good solvent. Only MBA-WSP was adjusted by a power law since it is the population with the clearest and greatest variation. The value of the power law exponent was found to be a = 0.27, which is very close to the theoretical value for dendrimers. The last result confirms the initial hypothesis consisting in that the water-soluble polymer formed during the synthesis is for both crosslinkers a hyperbranched polymer of diameter of 30 to 60 nm.

### C) Spectromechanical Characterization of Films Comprising Microgels Alone (Comparative), Water-Soluble Polymers Alone (Comparative) and Films Comprising Microgels and WSP (Invention)

These films are formed from the aqueous suspension comprising MG and WSP which is obtained from the polymerization process by aqueous precipitation, before any centrifugation step.
F6 - 2 mol% OEGDA: (32 wt% WSP - 68 wt% MG);
F7 - 8 mol% OEGDA: (22 wt% WSP - 78 wt% MG);
F6' - 2 mol% MBA: (29 wt% WSP - 71 wt% MG);
F7' - 8 mol% MBA: (20 wt% WSP - 80 wt% MG);

Other films are formed by mixture with a controlled ratio of microgel (MG) and water-soluble polymer (WSP) which have been collected from the precipitate and the supernatant material after the centrifugation step.
F1 - 2 mol% OEDGA comparative (0 wt% WSP - 100 wt% MG);
F1' - 2 mol% MBA comparative (0 wt% WSP - 100 wt% MG);
F2 - 2 mol% OEDGA (25 wt% WSP - 75 wt% MG);
F2' - 2 mol% MBA (25 wt% WSP - 75 wt% MG);
F3 - 2 mol% OEDGA (50 wt% WSP - 50 wt% MG);
F3' - 2 mol% MBA (50 wt% WSP - 50 wt% MG);
F4 - 2 mol% OEDGA (75 wt% WSP - 25 wt% MG);
F4' - 2 mol% MBA (75 wt% WSP - 25 wt% MG);
F5 - 2 mol% OEDGA comparative (100 wt% WSP - 0 wt% MG);
F5' - 2 mol% MBA comparative (100 wt% WSP - 0 wt% MG);

### 1. Spectromechanical Characterization Method

To study the mechanical properties, the films are characterized by linear oscillatory rheology and non-linear extensional rheology on an MCR 302 rheometer from Anton Paar.

Oscillatory rheology is performed on films of thickness of around 1 mm with parallel plates of 8 mm at controlled temperature. The frequency sweeps are performed at 20° C. from 0.01 to 600 rad/s at constant elongation of 1% which ensures the linear regime.

The extensional rheology is performed by using a Sentmanat extension rheometer (SER) geometry which consists of paired windup drums moving in equal but opposite rotation. The tests are conducted at 20° C. The film dimensions are found in the following interval: 0.5-1 mm x 1-2 mm x 15-20 mm. The thickness and width are respectively measured before the test by light microscopy and compass. Films are stretched to break with a constant elongation rate, also referred to as the Hencky elongation rate εH. A minimum of fifty samples is tested for each type of film. The extensional viscosity ηE is measured as a function of time. The logarithmic elongation in the sample, also referred to as the Hencky elongation εH, is a function of ε, the constant elongation rate; t, time; L, the sample length at time t and L0, the initial sample length. In order to calculate the elongation at break εR, the constant elongation rate is multiplied by the time to break.

### 2. Spectrophotometric Characterization of the Comparative Films (F1: 0% WSP and 100% MG)

The impact of temperature on formation was evaluated by forming purified films at 20° C., 32° C., 37° C. and 60° C. In this domain, the goal was to evaluate whether the state of the microgels, i.e., collapsed or swollen, impacts the quality of assembly during film formation. Indeed, during the liquid-to-solid transition, one can imagine that the state of the microgel can play an important role, i.e., that swollen microgels under the VPTT would create more intra-tangles than collapsed microgels above the VPTT The extensional viscosity was measured at 20° C. with a constant elongation rate of 0.5 s⁻¹.

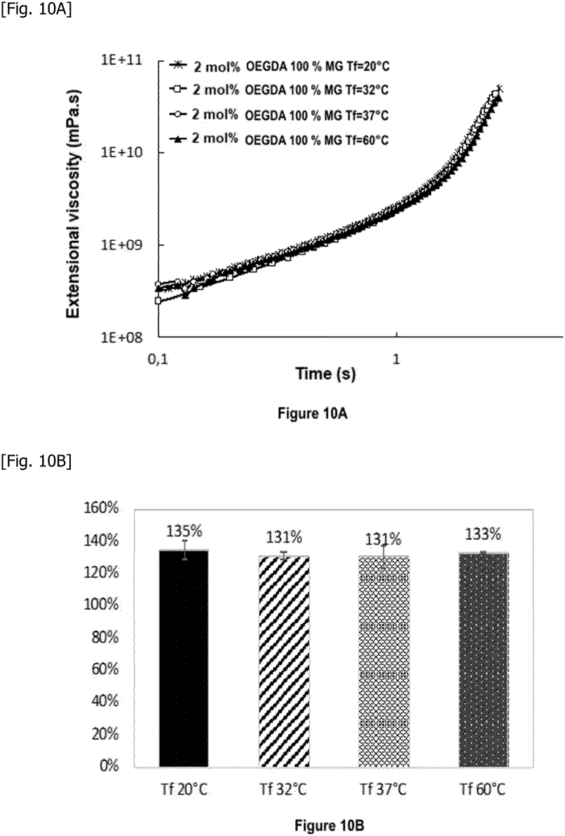

FIG. 10 presents the mean extensional viscosity for four different formation temperatures. It is clearly observed that the formation temperature does not impact the extensional viscosity or the elongation at break. It is not a parameter controlling the mechanical properties of the films. If the microgels are under the VPTT (20° C.), in the interval (32° C., 37° C.) or above (60° C.), the elongation at break remains 133 plus or minus 2%. The microgel shell appears to retain some mobility at 60° C. which allows the microgels to interpenetrate one another in their collapsed state. Moreover, the microgel films, once formed, are maintained at 20° C. and tested at 20° C. The chains then have the time to relax and reach a similar state of interpenetration independently of the formation temperature.

### 3. Spectromechanical Characterization of the Films (F6 and F7)

The following four systems were studied: 2 mol% OEGDA, 2 mol% MBA; 8 mol% OEGDA and finally 8 mol% MBA. FIG. 4 indicates the ratio of soluble polymer in water and the microgels of the different films which have been prepared. 3.1 Linear rheology: results

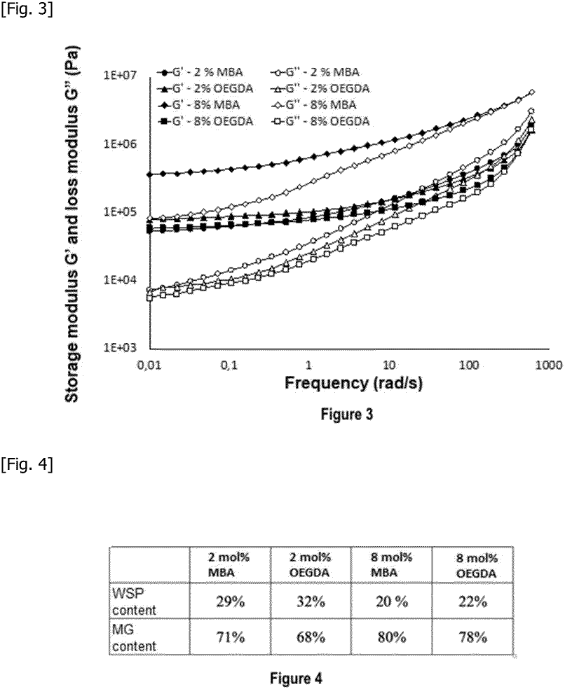

The dynamic rheology response is compared for the different films in FIG. 3. Frequency sweeps, at fixed aspect ratio in the linear domain, were performed at 20° C.

All the films generally behave as viscoelastic solids since G' is greater than G" at low frequency values. The films reach a plateau with low frequency values, which is characteristic of the entanglements of chains in the films (elastic network). The transition toward the vitreous region is observed in all cases by an intersection point of G' and G" at higher frequencies. The storage moduli for 2 mol% OEGDA and 2 mol% MBA are around 1 x 10⁵ Pa at 1 Hz (6 rad/s). The films thus have a very low stickiness. Indeed, the Dahlquist criterion states that G′ would be less than 1 x10⁵ Pa at 1 Hz for the material to have a fast and measurable stickiness.

The modulus of the 8 mol% MBA films increases considerably in comparison to the 2 mol% crosslinker, respectively around 1.5 x 10⁶ Pa and 1 x 10⁵ Pa at 1 Hz (6 rad/s). The increase in conservation modulus, arising from the higher crosslinker density, leads to a less flexible film, which exhibits no tackiness at all.

An 8 mol% OEGDA film surprisingly demonstrates very similar moduli to 2 mol% films. This particular behavior could be explained by a higher quantity of water in the film at the time of the test, due to the uncontrolled hygrometry environment or to incomplete drying of the film.

### 3.2 Extensional Rheology: Results

Extensional rheology consists of the elongation of the material in the non-linear domain of deformations, at a constant elongation rate. Uniaxial extension can produce a much greater degree of molecular orientation and stretching than simple tearing. Consequently, extensional rheology is more sensitive to branching of the long polymer chain and can be more descriptive than other types of rheological testing by mass. Extensional rheology allows establishing a relationship with adhesive behavior.

The extensional viscosity of OEGDA and MBA films for 2 mol% and 8 mol% of crosslinker was measured at 20° C. at a constant elongation rate: 0.5 s⁻¹. The elongation at break logarithm εR and the L_{R/}L₀ ratio were calculated with the following formula: ε_{R} = ε x t_{R} = ln (L_{R}/L₀).

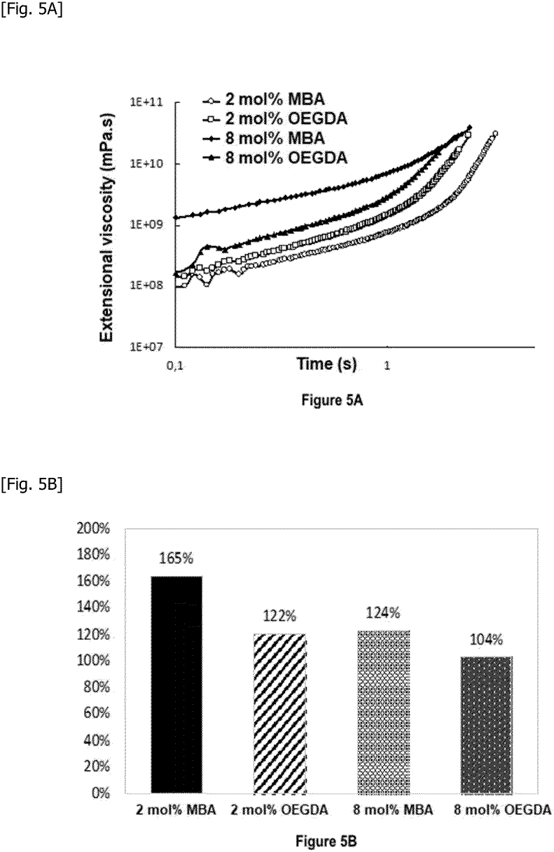

FIG. 5 (right) presents the mean extensional viscosity as a function of time for MBA and OEGDA for both amounts of crosslinker. First, the extensional viscosity permanently increases by following the linear viscoelastic envelope defined by the slope of 3 times the complex viscosity: 3.η*.

For elongation values greater than 40%, the extensional viscosity starts to deviate upward from the shear viscosity at speed zero. This upward deviation is referred to as elongation-hardening and typically appears in chemically crosslinked or physically well-entangled polymers. It is an aspect indicative of the branching architecture of the chain. The chain entanglements begin to resist extension at each time that the chain extensibility limit is approached and cause the network to stiffen. Consequently, the extensional viscosity follows an upward deviation. The appearance of elongation-hardening was expected since the films respectively contain 2 mol% and 8 mol% of crosslinker. Elongation-hardening was considered to be a desirable property for better adhesive performance. It fulfills the typical requirement for an adhesive that fails without leaving a sticky residue on the surface by providing network cohesion at high deformation.

The OEGDA and MBA films have a similar behavior in the linear region. The elongation at break is significantly higher, however, for MBA than OEGDA independently of the crosslinker density, FIG. 5 (left). This indicates that the MBA network resists higher stress before failure, although it does not differentiate the influence of the water-soluble polymer and the microgels. The increase of the crosslinking density leads to a lower elongation at break for both crosslinkers. Indeed, a more crosslinked network cannot stretch as much to accommodate the stress, and breaks at an earlier stage. Denser and more crosslinked particles also tend to interpenetrate less with each other, thus creating a weaker network.

### 4. Spectromechanical Characterization of the Films (F1 to F5)

The formulation is performed consisting of film formation with a controlled ratio of WSP and MG for 2 mol% MBA films and 2 mol% OEGDA films. The method of linear oscillatory rheology and linear extensional rheology is identical to that described above.

### 4.1 Linear Rheology: Results

For the two crosslinkers OEGDA and MBA, frequency sweeps were performed at 20° C. on the five different films formulated, i.e., with an MG content of 0% by mass to 100% by mass.

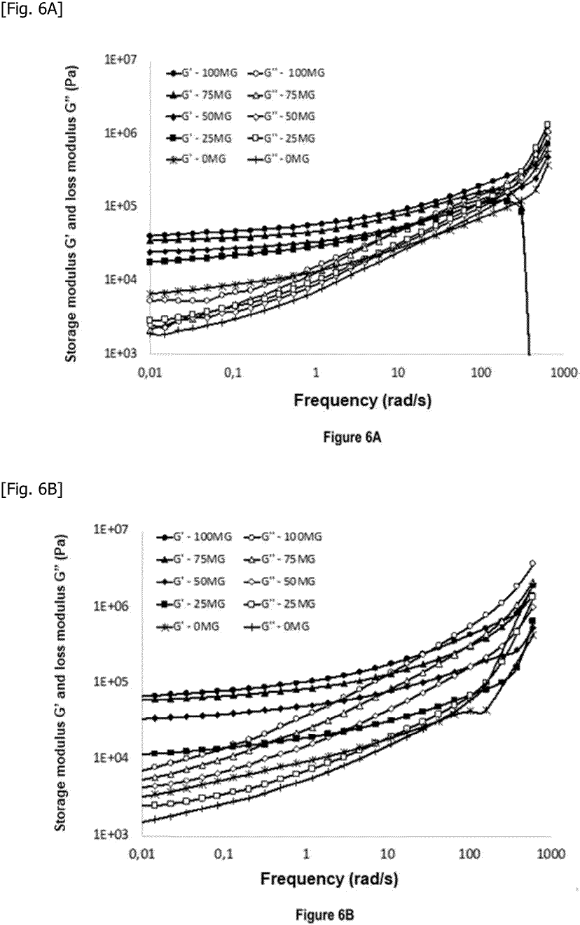

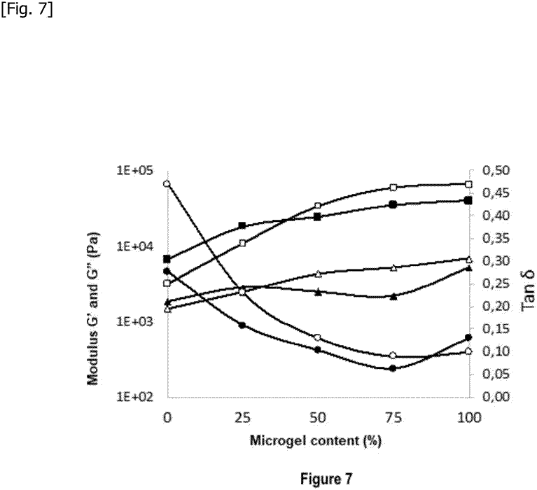

The dynamic rheology response is compared for the different MG contents for OEGDA and MBA in FIG. 6. First of all, it is important to note that all the films, independently of the MG content, behave as viscoelastic solids since G' is greater than G" at low frequency values. Independently of the MG content, all the microgel films have reached a plateau at low frequencies, which indicates the presence of a polymer network The flow region was not observed, even for films consisting of 100% WSP. This important result indicates crosslinking and/or branching in the water-soluble polymer. Thus, WSP is not linear and does not flow like thermoplastic polymers (in the scales of the parameters explored in this document, frequency, temperature, etc.). Glass transition is observed in all the films by an intersection point of G' and G" at high frequencies. For both crosslinkers, growth does not significantly vary with the addition of WSP. Glass transition thus appears at approximately the same frequency independently of the amount of WSP. This is not surprising since the glass temperature of a polymer is determined by its molecular structure. As observed in FIG. 7, the storage and loss moduli (taken at the rubbery plateau at ω = 0.01 rad.s⁻¹) increase significantly with the increase in MG content. MG particles increase the shear modulus, similar to fillers that consolidate a composite. Moreover, tan δ, the ratio of G" to G' decreases for an increasing MG content, which indicates that the dissipative response of the films is lesser and less pronounced. The ability to deflect the shear modulus to higher or lower values by controlling the MG/WSP ratio is extremely valuable since it provides a simple lever to adjust the tacky or adhesive properties of films depending on the targeted application. For both systems, G' is just about 1 x 10⁵ Pa at 1 Hz: 8.5 x 10⁴ Pa and 1.5 x 10⁵ Pa, respectively, for OEGDA-MG and MBA-MG. It is indicated that the films have a very fine stickiness at 20° C. when they are completely purified.

### 4.2 Extensional Rheology: Results

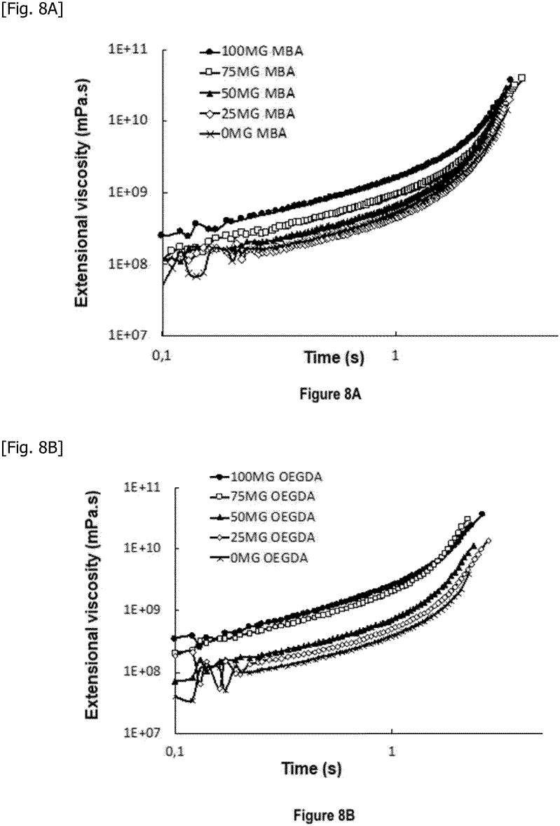

The extensional viscosity results were measured for the 2 mol% OEGDA and MBA films with a different MG content at 20° C. for constant elongation rate: 0.5 s⁻¹. FIG. 8 presents the mean extensional viscosity as a function of time for MBA and OEGDA for different MG contents.

For elongation values greater than 40%, the extensional viscosity starts to deviate upward from the shear viscosity at speed zero for all formulations. As previously mentioned, this upward deviation is called elongation-hardening. The appearance of elongation/hardening for films of 100% MG is expected since the films contained 2 mol% crosslinker providing crosslinked points to the network. However, it is more surprising to note that all the films, including 100% WSP, present elongation-hardening. Consequently, we can suppose that the stiffening of the network is much more bound by the chemical crosslinking points than the physical entanglements of branched chains. As expected, the microgel content has a significant impact on the extensional viscosity values. Indeed, the microgels act as fillers stiffening the matrix and increasing the G' and G" moduli. As a direct consequence, the higher moduli increase the extensional viscosity value.

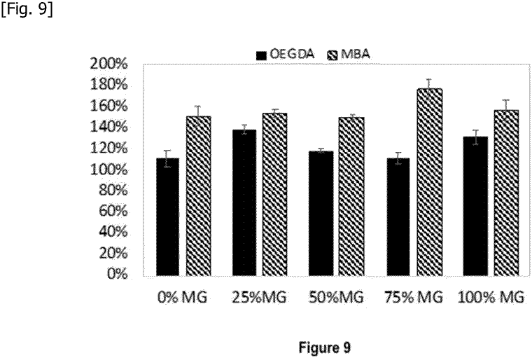

However, it results from this that the microgel content does not impact either the elongation-hardening or the elongation at break. In the field of future applications, this result is extremely positive since it indicates that the proportion of water-soluble polymer can be modified to adjust the desired modulus according to the application, but without in any case losing the stretch capacity of the film. As observed in FIG. 9, the logarithmic elongation at break is significantly higher for MBA films than for OEGDA independently of the microgel content. This first confirms that the MBA microgel network resists a higher stress before break than the OEGDA particles. This reinforces the supposition of a higher capacity of MBA to form a more solid network, this being surely due to a “looser shell” architecture for this latter than for OEGDA-MG. The second important result from FIG. 9 consists in that MBA films consisting of 100% WSP also have a higher elongation at break than OEGDA-WSP films. This corroborates the SEC results which show a more branched structure in the case of MBA-WSP.

### D) Assembly of the Microgel (F1 to F5)

Method: Topographical images were captured by AFT (Bruker Multimode 8 device) to analyze the assembly of microgels on the surface of the film. PeakForce QNM Air mode and ScanAsyst Air probes (average spring constant k of 0.4 N.m⁻¹) were used for all scans. Sharp cross-sections of microgel films were prepared to visualize the assembly inside the film with a Leica EM UC7 cryo-ultramicrotome, by using a Leica EM-FC7 cryochamber cooled to -80° C. Finally, the films were manually subjected to unidirectional stretching and images were formed in the stretched state. The elongation was 30%.

### Results for the Comparative Films F1 and F1

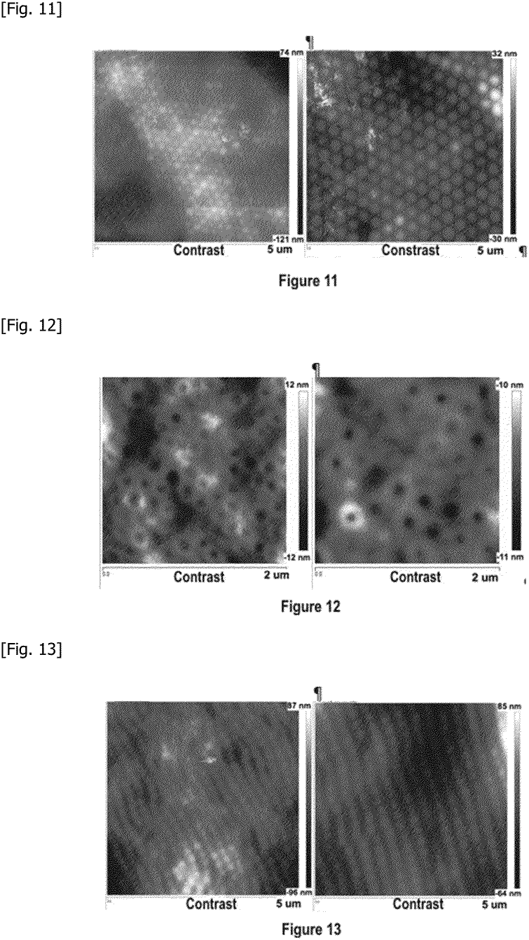

Atomic force microscopy was performed on the upper surface of films created with 100% microgels (no water-soluble polymer). The microgel particles self-assemble into perfect hexagonal close packing as seen in topographic contrast images in FIG. 11 (right) for microgels of 2 mol% OEGDA-crosslinked and (left) for microgels of 2 mol% MBA-crosslinked. Unlike conventional latex, the particles do not coalesce but maintain their spherical form with a certain interpenetration with one another. The AFM topographical contrast images showed the slightly larger size of the MBA-crosslinked microgels.

The film cross-sections were surface treated by ultra cryomicrotomy and observed by AFM. The topographical contrast images in FIG. 12 show the packing of spherical particles with localized hexagonal packing. Unlike the deformation of particles having fallen and dried on a hard wafer, the multiple layers of MG stacking during solvent evaporation did not flatten and remained spherical. A contrast difference of around 15 nm between the core and the shell is observed for the majority of microgels in both types of crosslinkers. Even after having been formed, the microgel films contain and absorb water from their environment. As a result, it can be suggested that the shell, which is much more swollen, is loosely crosslinked and the core, which is hollower, is more crosslinked than the shell.

The microgel films were stretched to obtain 30% elongation and their upper surfaces were observed by AFM. FIG. 13 shows a topographical contrast image of 5 mm of OEGDA-crosslinked and MBA-crosslinked microgel films. The direction of stretching is parallel to the X axis. Stretching causes loss of compact hexagonal packing and deformation of the particle network. Spaces appear between the microgels in the direction of deformation. However, it seems that the microgels are not significantly deformed and maintain their spherical form. It is suggested that, at this elongation, the majority of tangled chains of the microgel shell stretch to accommodate the stress and the dense core is not yet deformed.

### Results of the Assembly of the WSP-MG Films According to the Invention F2 to F4

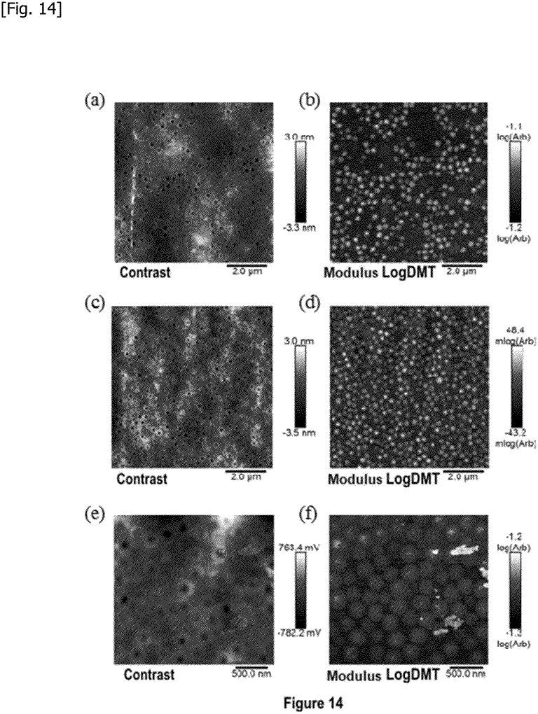

The cross-sections of the reformulated films with controlled microgel content were observed by AFM. FIG. 14 presents the topographic contrast image of 2 microns (a) and the logDMT modulus (b) of 2 mol% MBA-MG films for 25 wt% of MG. The logDMT modulus channel shows the stiffest regions in lighter colors and the more flexible ones in darker colors. As you would expect, the microgels are much denser than the water-soluble polymer and resemble fillers dispersed in a flexible composite matrix. They do not form aggregates but rather are perfectly homogeneously dispersed. FIG. 14 presents the topographic contrast image of 2 microns (c) and the logDMT modulus (d) of 2 mol% MBA-MG films for 50 wt% of MG. The microgels start to be in contact with one another but no structured arrangement is observed. In certain areas, microgel depletion is observed. FIG. 14 presents the topographic contrast image of 500 nm (e) and the logDMT modulus (f) of MBA-MG films for 75 wt% of MG. Similar conclusions can be expressed; the microgels are the closest but no hexagonal packing arrangement is observed.

Once again, the core/shell structure is clearly observed with a clear gradient of the modulus between the core relative to the WSP matrix. The denser and less swollen cores are characterized by hollows, the shells are more swollen than the cores but slightly denser than the water-soluble polymer which is characterized by the lower modulus. A very gradual transition is observed between the microgel shell and the water-soluble polymer, which suggests a similar structure as well as a certain interpenetration between the two phases.

### E) Conclusion

The linear rheology on the 2 mol% OEGDA and MBA-crosslinked microgel films shows that the shear modulus value can vary and be adjusted by means of the water-soluble polymer content, from 0% to 100%. As a result, a range of films can be formed from very sticky to films with no stickiness on any substrate by simple evaporation of water. The microgel films display interesting mechanical properties at high deformation. The elongation at break goes up to 60%, demonstrating that these films have a solid cohesion for a network of simple and spontaneous self-assembled particles. Moreover, the variation of the water-soluble polymer makes it possible to vary the rheological properties without changing the stretch properties of the films. Indeed, all the films, independently of the microgel content, have an elongation-hardening, which is known as being a property of value for adhesives since it allows the cohesion of a film during detachment. The latest discovery suggests that a water-soluble polymer is obviously branched and/or crosslinked since films completely composed of the latter also exhibit elongation-hardening. The elongation at break of the MBA-crosslinked films is significantly higher than the OEGDA-crosslinked films for 2 mol% of crosslinker. This is in correlation with the results on the suspension viscosities which show that the MBA-crosslinked particles have a higher interaction and tend to create a more solid network. These more solid particles, probably due to the higher number of chains suspended on the surface, lead to a higher elongation at break when the film is formed since the microgels are more interpenetrated with one another. Moreover, the elongation at break decreases for a higher crosslinker ratio in a similar way that the viscosity decreases for dispersions. Denser particles tend to have less interpenetration and form a weaker network than more loosely crosslinked particles.

1-16. (canceled)

17. Cosmetic composition, pharmaceutical composition or medical device each comprising a water-soluble polymer and microgel particles, said microgel particles having an average size of 100 nm to 1000 nm in a dry state,
wherein the microgel particles and the water-soluble polymer may be independently obtained by aqueous phase precipitation polymerization,in the presence of a crosslinking agent, of at least following three monomers, said monomers being:
di(ethylene glycol) methyl ether methacrylate,
an oligo(ethylene glycol) methyl ether methacrylate having a number average molar mass from 400 g/mole to 600 g/mole, and
a vinyl monomer comprising a carboxyl group,
wherein at least one of the two crosslinking agent is N,N′-methylenebisacrylamide,
wherein a ratio between a mass of the water-soluble polymer and a mass of the microgel particles in a dry state is between 0% and 100% by mass,
wherein the medical device includes a mixture consisting of the water-soluble polymer, the microgels and optionally water, and
wherein a solid content of the cosmetic composition, a solid content of the pharmaceutical composition and a solid content of said mixture are from 1.5% to 100% by mass.

18. Cosmetic composition, pharmaceutical composition or medical device according to claim 17, wherein the cosmetic composition, the pharmaceutical composition or the mixture contains from 50% to 100% by mass of a mixture consisting of microgel particles and water-soluble polymer and from 0% to 50% by mass of water, the percentages being relative to a mass of the composition, and a mass of the microgel particles being the mass of the microgel particles in a dry state.

19. Cosmetic composition, pharmaceutical composition or medical device according to claim 17, wherein the solid content of the cosmetic composition, the solid content of the pharmaceutical composition and the solid content of the mixture are from 50% to 100% by mass.

20. Cosmetic composition, pharmaceutical composition or medical device according to claim 17, being in the form of a film which has a thickness of 500 microns to 1000 microns.

21. Cosmetic composition, pharmaceutical composition or medical device according to claim 17, wherein a radius of gyration of the water-soluble polymer measured by size exclusion chromatography at 20° C. is from 5 nm to 80 nm.

22. Cosmetic composition, pharmaceutical composition or medical device according to claim 17, wherein the water-soluble polymer has an average molecular weight measured by steric exclusion chromatography at 20° C. of from 1 × 10⁵ g.mol⁻¹ to 1 × 10⁶ g.mol⁻¹.

23. Cosmetic composition, pharmaceutical composition or medical device according to claim 17, wherein the mole fraction of di(ethylene glycol) methyl ether methacrylate is from 80 mol% to 90 mol%, the mole fraction of oligo(ethylene glycol) methyl ether methacrylate is from 5 mol% to 15 mol%, the mole fraction of the carboxyl-bearing vinyl monomer is from 2 mol% to 8 mol%, and the mole fraction of the crosslinking agent is from 0.5 to 10 mol%, the sum of the four mole fractions being equal to 100% by mole.

24. Cosmetic composition, pharmaceutical composition or medical device according to claim 17, wherein the ratio between the mass of the water-soluble polymer and the mass of the microgel particles is between 0% and 35%.

25. Cosmetic composition, pharmaceutical composition or medical device according to claim 17, wherein the ratio between the mass of the water-soluble polymer and the mass of the microgel particles is between 35% and 100%.

26. Topical cosmetic treatment method of skin, nails, lips, mucosa or hair of a person, said method comprising a first step of applying onto the person a cosmetic composition according to claim 17.

27. Process for preparing a cosmetic composition, a pharmaceutical composition or a medical device according to claim 17, said process comprising a step of preparing a mixture of microgel particles and a water-soluble polymer, said step comprising a first step and a second step which can be successive in any order, or simultaneous:
said first step being a step of preparing the microgel particles and comprising a step of aqueous phase precipitation polymerization, in the presence of a crosslinking agent, of at least three monomers,said monomers being:
di(ethylene glycol) methyl ether methacrylate,
an oligo(ethylene glycol) methyl ether methacrylate,
a vinyl monomer bearing a carboxyl group,
and a purification step to recover microgel particles which have been obtained from the step of aqueous phase precipitation polymerization,
said second step of preparing the water-soluble polymer, and comprising a step of aqueous phase precipitation polymerization, in the presence of a crosslinking agent, of at least three monomers, said monomers beingt:
di(ethylene glycol) methyl ether methacrylate,
an oligo(ethylene glycol) methyl ether methacrylate,
a vinyl monomer bearing a carboxyl group,
and a purification step to recover the water-soluble polymer which has been obtained from the step of aqueous phase precipitation polymerization,
wherein at least the first or the second crosslinking agent is N,N′-methylenebisacrylamide,
a third step which follows said first step and said second step, said third step being a step of mixing a mass of purified microgel particles which are obtained from the first step and a mass of water-soluble polymer which is obtained from the second step,
wherein the ratio between the mass of the water-soluble polymer and the dry mass of the microgel particles is between 0% and 100%.

28. Process according to claim 27, wherein the purification step in the first stepcomprises at least one centrifugation/redispersion cycle to separate a precipitate and a supernatant material, and a step of recovering the microgel particles in the precipitate.

29. Process according to claim 27, wherein the purification step in the second step comprises at least one centrifugation/redispersion cycle to separate a precipitate and a supernatant material, and a step of recovering the water-soluble polymer in the supernatant material.

30. Process according to claim 27, wherein the first step and the second step are a single step, such that the microgel particles and the water-soluble polymer are produced at the same time from the same monomers, the single step comprising at least one centrifugation/redispersion cycle for separating the precipitate and the supernatant material which have been obtained from the aqueous phase precipitation polymerization of the monomers, said microgel particles being recovered in the precipitate and said water-soluble polymer being recovered in the supernatant material.

31. Process according to claim 30, wherein - in the single step - the ratio between the mass of the water-soluble polymer in the supernatant material and the mass of the microgel particles in the precipitate is a first ratio and - in the third step - the ratio between the mass of the water-soluble polymer and the mass of the microgel particles is a second ratio which is between 0% and 100% and which is different from the first ratio.

32. Process according to claim 27, wherein the first crosslinking agent and/or the second crosslinking agent are N,N′-methylenebisacrylamide.
